(19) United States
(12) Patent Application Publication
(10) Pub. No.: US 2012/0149005 A1
(43) Pub. Date: Jun. 14, 2012

(54) REACTION TREATMENT DEVICE AND REACTION TREATMENT METHOD

(75) Inventors: Kensuke Kojima, Kanagawa (JP); Hiroshi Katayama, Kanagawa (JP); Akira Shinozaki, Tokyo (JP); Akira Toda, Kanagawa (JP); Yoshiaki Kato, Gunma (JP); Hidetoshi Watanabe, Chiba (JP); Tasuku Yotoriyama, Tokyo (JP); Junji Kajihara, Tokyo (JP); Toshio Watanabe, Kanagawa (JP); Masahiro Miyachi, Tokyo (JP); Takanori Anaguchi, Kanagawa (JP)

(73) Assignee: Sony Corporation, Tokyo (JP)

(21) Appl. No.: 13/294,443

(22) Filed: Nov. 11, 2011

(30) Foreign Application Priority Data
Nov. 12, 2010 (JP) 2010-254157
Oct. 7, 2011 (JP) 2011-223317

(51) Int. Cl. C12Q 3/00; C12M 1/38

(52) U.S. Cl. 435/3; 435/286.1

(57) ABSTRACT

Disclosed herein is a reaction treatment device including a temperature control section which includes a first temperature control section disposed at an outer peripheral edge part of a group of reaction regions and a planar second temperature control section, wherein the first temperature control section and the second temperature control section are disposed opposite to each other, with the reaction region group therebetween.

### CROSS REFERENCES TO RELATED APPLICATIONS

The present application claims priority to Japanese Patent Application No. 2010-254157 filed on Nov. 12, 2010 and Japanese Patent Application No. 2011-223317 filed on Oct. 7, 2011, the disclosures of which are incorporated herein by reference.

### BACKGROUND

The present disclosure relates to a reaction treatment device and a reaction treatment method. More particularly, the present disclosure relates to a reaction treatment device and a reaction treatment method by which high-accuracy temperature control can be performed.

In the cases where reaction should be controlled based on temperature conditions, it is desirable to control the temperature conditions with higher accuracy, irrespectively of whether the reactants are liquid, solid or gas. This desire exists also in such technical fields as gene analysis, for example.

As an example, in various fields of biotechnology, a technique of amplifying a specific nucleic acid, such as the PCR (Polymerase Chain Reaction) method for performing gene amplification, is applied, to be utilized as a nucleic acid detection method.

As a nucleic acid detection method, there has been known a method in which a hybridization probe labeled by a fluorescent substance is used. As variations of the nucleic acid detection method, there have been known, for example, a nucleic acid determination method (real-time PCR method) and a detection method (melting curve analysis) for mutation such as single nucleotide polymorphism (SNP). If the SNP analysis can be carried out speedily and easily, it will be possible to perform, for example, tailor-made medical care in which optimum therapeutics, medication and the like are diagnosed at the patient's bedside, which becomes a promising POC (Point Of Care). In view of this, there is a request for a method of checking the nucleic acid amplification after a nucleic acid amplification reaction in a more speedy and easy manner.

Besides, as the detection method, there have also been known a method in which amplification is checked by measuring the turbidity of the reaction mixture having been served to a nucleic acid amplification reaction and a method based on the use of a microarray having a probe which is coupled specifically with an object nucleic acid of amplification.

Meanwhile, the main stream of reaction detection is, in general, a reaction detection carried out by a system having a large-scale heat block while using, for example, a 96 multiassay plate supplied with prepared reagents (see Japanese Patent Laid-open No. 2006-162625). This system, however, is disadvantageous in that the heat capacity is great because uniform heating is adopted, a large amount of electric power is therefore consumed in the heat-generating section, and a correspondingly long time is taken for cooling. For these reasons, this system would fall far short of a portable system, in practice.

Furthermore, there have been a number of precedents for a heating reaction performed by use of a microchip, examples of which include a system in which a heater wire is wound around a capillary tube and a system in which four heat rods are put into contact with the four corners of a microchip.

For example, a system in which a chip is simply sandwiched between upper and lower two flat plates has been known, as exemplified by the device described in Japanese Patent Laid-open No. 2009-300299. In addition, there is also known a system leading easily to an idea of a heater structure in which a heater is put into contact along a shape corresponding to channels or reaction sites, as exemplified by the reaction device described in Japanese Patent Laid-open No. 2008-253227.

### SUMMARY

However, the above-mentioned reaction treatment devices in the past are unsatisfactory in that temperature distribution varies greatly depending on the design of the structures surrounding the reaction sites. In addition, each time the shape of the reaction sites is changed, it is necessary to fabricate and set a heater or heaters fitting to the shape thus changed. This makes it necessary to perform tunings in relation to electric power and control each time of such a change. This is very bothersome.

In other words, all of the above-mentioned configurations in the past are unsatisfactory for making uniform the temperature distribution, so that it is very difficult for the configurations to guarantee stability of reaction sites.

Besides, where the number of reaction site wells is increased, it is necessary to process a heater or heaters on the basis of each chip design and to design a temperature distribution each time of chip change. This means poor versatility.

Thus, there has been a need for a reaction treatment device and a reaction treatment method by which temperature control can be made easily and with high accuracy.

According to an embodiment of the present disclosure, there is provided a reaction treatment device including a temperature control section which controls temperature of an outer peripheral edge part of a group of reaction regions.

Preferably, the temperature control section is a first temperature control section disposed at the outer peripheral edge part of the group of reaction regions, the reaction treatment device includes a reaction temperature control section including the first temperature control section and a planar second temperature control section, and the first temperature control section and the second temperature control section are disposed opposite to each other, with the reaction region group therebetween.

Preferably, the first temperature control section is rectangular frame-like in shape.

Preferably, the group of reaction regions is disposed in a substrate, and the first temperature control section and the second temperature control section make contact with the substrate.

Preferably, edges of an outer peripheral part and/or an inner peripheral part of a frame body portion of the first temperature control section are each provided with a single or a plurality of cutouts.

Preferably, the cutout or cutouts are provided in corners of the outer peripheral part and/or in central parts of the edges of the inner peripheral part.

Besides, preferably, the temperature control section is flat plate-like in shape and has a light-transmitting opening section at a part corresponding to each reaction region in the group of reaction regions (hereinafter, this temperature control section will be referred to also as “the temperature control section having the opening section”).

Preferably, the group of reaction regions is disposed in a substrate, and the temperature control section having the opening section makes contact with the substrate.

Preferably, the temperature control section having the opening section is disposed between the substrate and a heat insulating section which restrains release of heat from the temperature control section.

Preferably, the temperature control section having the opening section has the opening section formed in a light-screening body, and the reaction treatment device further includes an irradiation section configured to irradiate the reaction regions with light and a detection section configured to detect light coming from the reaction regions.

Preferably, the reaction treatment device includes two above-mentioned temperature control sections having the opening section, and the two temperature control sections are disposed opposite to each other, with the reaction region group therebetween.

According to another embodiment of the present disclosure, there is provided reaction treatment method wherein temperature of an outer peripheral edge part of a group of reaction regions is controlled by a temperature control section disposed at least at the outer peripheral edge part, whereby temperature of the reaction region group is controlled.

Preferably, the temperature control section is a first temperature control section disposed at the outer peripheral edge part of the reaction region group,

the first temperature control section and a planar second temperature control section are disposed opposite to each other, with the reaction region group therebetween, and the first temperature control section and the planar second temperature control section cooperate with each other so as to control temperature of the reaction region group.

Preferably, a single or a plurality of cutouts are provided in an outer peripheral part and/or an inner peripheral part of a frame body portion of the first temperature control section so as to suppress local heating.

In addition, also preferably, temperature of the reaction region group is controlled by suppressing local heating in the reaction region group while controlling temperature of the outer peripheral edge part by a flat plate-like temperature control section which has a light-transmitting opening section at a part corresponding to each reaction region in the reaction region group.

According to embodiments of the present disclosure, there are provided a reaction treatment device and a reaction treatment method by which temperature control can be made easily and highly accurately.

Additional features and advantages are described herein, and will be apparent from the following Detailed Description and the figures.

### BRIEF DESCRIPTION OF THE FIGURES

FIG. 1 is a conceptual diagram of a reaction treatment device according to an embodiment of the present disclosure;

FIG. 2 is an exploded perspective view showing a major part of the reaction treatment device;

FIGS. 3A to 3D are top views of a frame body portion of a first temperature control section according to an embodiment of the present disclosure and modifications of the frame body portion;

FIG. 4 shows schematic views of reaction treatment devices (provided respectively with a lower-surface heater only, the lower-surface heater plus an upper-part heater A, the lower-surface heater plus an upper-part heater B, the lower-surface heater plus an upper-part heater C, and the lower-surface heater plus an upper-part heater D) and temperature distributions during operations of the reaction treatment devices;

FIG. 5 shows a reaction treatment device (provided with the upper-part heater B) according to an embodiment of the present disclosure, its modifications (provided with the upper-part heaters C and D, respectively), and a reaction treatment device (provided with the upper-part heater A) according to the related art;

FIG. 6 is a schematic view of a working example of the reaction treatment device according to an embodiment of the present disclosure;

FIG. 7 is a conceptual diagram of a reaction treatment device according to an embodiment of the present disclosure;

FIG. 8A is a top view, and FIG. 8B a sectional view, of a temperature control section according to an embodiment of the present disclosure;

FIG. 9 is a conceptual diagram illustrating propagation of rays of light from a light source in the case where the temperature control section according to an embodiment of the present disclosure is used; and

FIG. 10 shows conceptual diagrams of reaction treatment devices (provided with an upper heater B only, an upper heater E only, and an upper heater F only, respectively) and temperature distributions during operations of the reaction treatment devices.

### DETAILED DESCRIPTION

Embodiments of the present application will be described below in detail with reference to the drawings.

Now, preferred embodiments of the present disclosure will be described below, referring to the drawings. Incidentally, the embodiments described below are merely representative examples of the embodiment of the present disclosure, and the present disclosure is not to be construed narrowly according to the embodiments.

### 1. Reaction Treatment Device of First Embodiment

(1) Group of reaction sites

(1.1a) Substrate

(2) Reaction temperature control section

(1.2a) First temperature control section

(1.2b) Second temperature control section

(3) Irradiation section

(4) Detection section

### 2. Nucleic Acid Amplification Reaction Device

(1) Nucleic acid amplification reaction

(2) Detection method for nucleic acid amplification (product)

### 3. Operation of Reaction Treatment Device of First Embodiment

### 4. Operation of Nucleic Acid Amplification Reaction Device Utilizing Reaction Treatment Device of First Embodiment

(1) Modifications

(4.1a) Operation of RT-LAMP device

(4.1b) Operation of RT-PCR device

### 5. Reaction Treatment Device of Second Embodiment

(1) Temperature control section

### 6. Operation of Reaction Treatment Device of Second Embodiment

### 7. Operation of Nucleic Acid Amplification Reaction Device Utilizing Reaction Treatment Device of Second Embodiment

### <1. Reaction Treatment Device of First Embodiment>

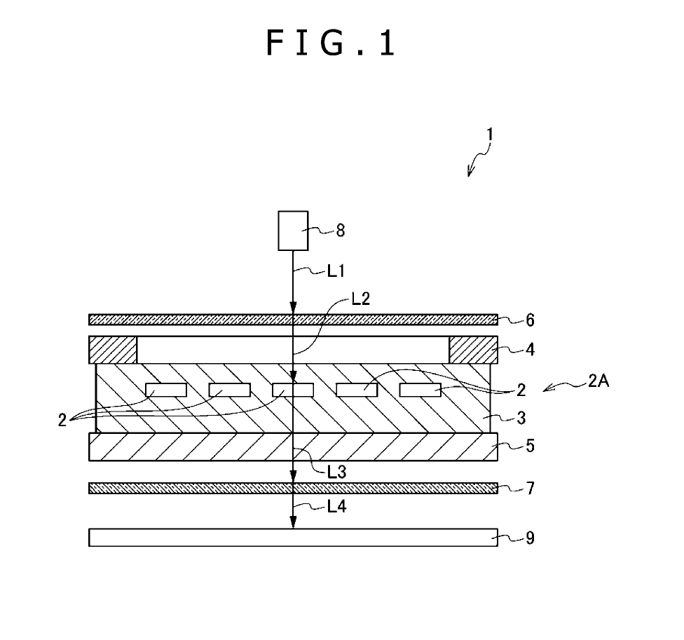

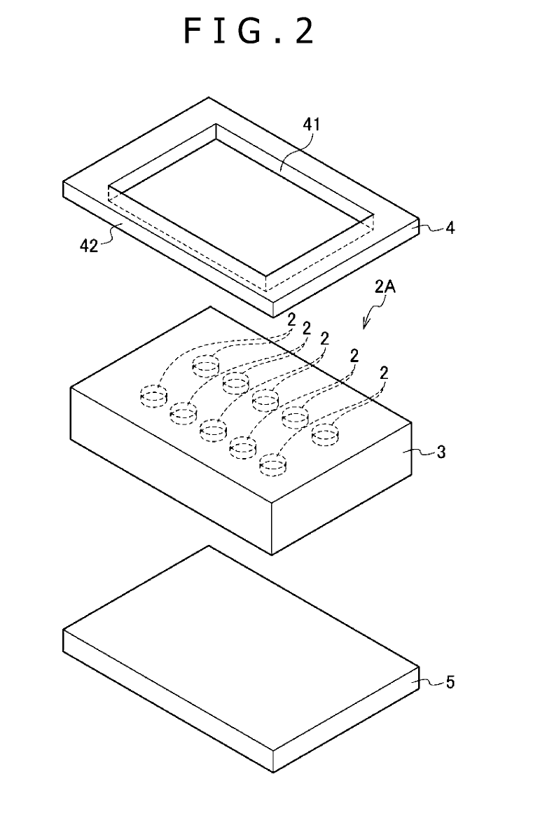

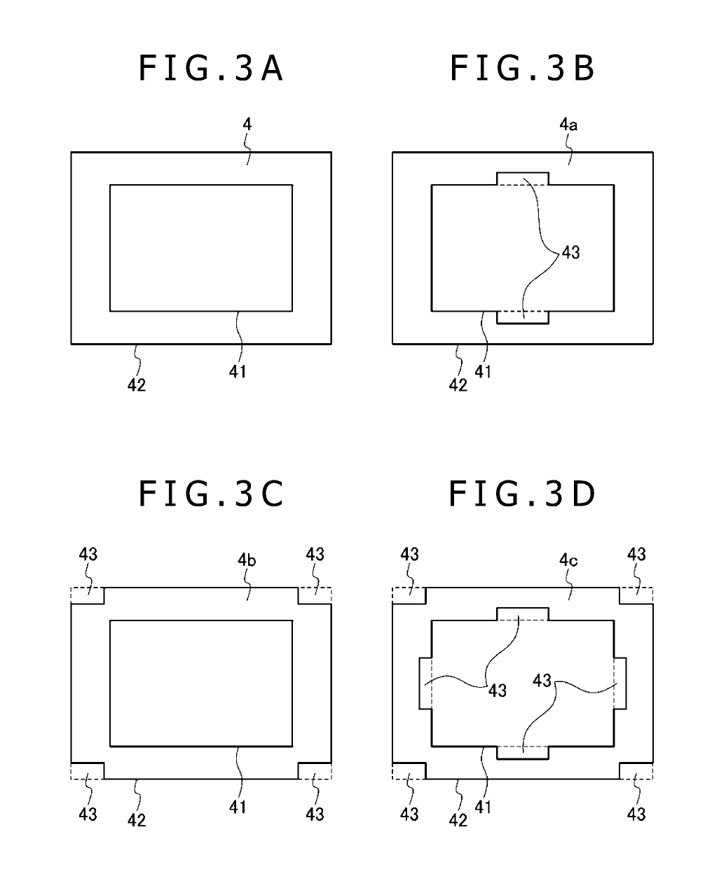

FIG. 1 is a conceptual diagram of a reaction treatment device 1 according to an embodiment of the present disclosure. In addition, FIG. 2 is an exploded perspective view of a major part of the reaction treatment device 1. FIGS. 3A to 3D are top views of a frame body portion of a first temperature control section 4 in the reaction treatment device 1 and modifications of the frame body portion.

Incidentally, in the drawings described below, the configurations of the device and the like are shown in simplified form, for convenience of description.

As shown in FIG. 1, the reaction treatment device 1 according to an embodiment of the present disclosure has at least a temperature control section 4 which controls the temperature of an outer peripheral edge part of a group of reaction regions (hereafter referred to also as “reaction region group”) 2A. The temperature control section 4 is a first temperature control section 4 disposed at the outer peripheral edge part of the reaction region group 2A, and the reaction treatment device 1 has a reaction temperature control section which includes the first temperature control section 4 and a planar second temperature control section 5. The first temperature control section 4 and the second temperature control section 5 are disposed opposite to each other, with the reaction region group 2A therebetween. Incidentally, the reaction region group 2A (substrate 3) can be mounted and dismounted.

Furthermore, the reaction treatment device 1 according to an embodiment of the present disclosure can be used also as an optical detection device or a nucleic acid amplification reaction device. For instance, as shown in FIG. 1, the reaction treatment device 1 is desirably further includes an irradiation section 8, an excitation filter 6, a fluorescence filter 7, and a detection section 9. As a specific example, a configuration may be mentioned in which at least the irradiation section 8 for irradiating reaction regions 2 with light and the detection section 9 for detecting light generated from the reaction regions 2 are provided.

In addition, though not shown, a configuration may also be adopted in which the light (scattered light, fluorescent light or the like) generated from the reaction regions 2 is reflected toward a detection section disposed, for example, on the side of the irradiation section 8 so that the light can be detected by the detection section.

Besides, though not shown, a pinhole, various filters, condenser lens, and support base may be appropriately disposed so as to support sections for controlling the quantity of light, light components and the like. In addition, preferably, a control unit (not shown) is provided for controlling the these components' various operations (e.g., light control, temperature control, nucleic acid amplification reaction, detection control, computation of quantity of light detected, monitoring, etc.).

Now, configurations of the reaction treatment device (nucleic acid amplification reaction device) 1 according to an embodiment of the present disclosure will be described in detail below.

### (1) Group of Reaction Regions

The reaction region group (area) 2A, which can be mounted and dismounted, is a section (region) in which a single or a plurality of reaction regions 2 to be reaction sites for various reactions are disposed.

The shape of the reaction site 2 is not specifically restricted, and examples thereof include a cylindrical shape and a quadrangular pyramidal shape.

Preferably, a plurality of the reaction regions 2 are arranged in the reaction region group 2A. The number of the reaction regions is not specifically restricted, and examples thereof include 6 (2 by 3, or the like) 25 (5 by 5, or the like), 24 (4 by 6, or the like), 96 (8 by 12, or the like) and 384 (16 by 24). According to the embodiment of the present disclosure, it is ensured that even when many reactions are made to take place simultaneously, the temperature distribution within the whole area of the reaction region group is little varied. In other words, the reaction region group as a whole can be heated substantially uniformly. Specifically, reactions in a uniform manner can be achieved, whereby detection accuracy and working efficiency are enhanced, which naturally is advantageous.

Incidentally, it suffices that object substances to be detected and substances necessary for the detection reactions are disposed in the reaction regions 2 appropriately. Examples of such substances include objects of detection which are derived from a biological body, synthetic oligomers (oligonucleotides, nucleic acid-like synthetic substances), synthetic oligomers obtained through modification of fluorescent dye or the like, enzymes, buffer solutions, salts, immobilizers such as wax, antigens, antibodies, solvents such as water, etc. Further, dNTP to be used in the PCR method, an isothermal amplification method or the like, dyes and other substances may also be disposed in the reaction regions 2 appropriately.

### (1.1a) Substrate (Microchip)

Preferably, a single or a plurality of the reaction regions 2 are formed as the reaction region group 2A in a mountable-and-dismountable reaction vessel (e.g., substrate) such as a microchip. Preferably, as shown in FIGS. 1 and 2, the reaction region group 2A is formed in the substrate 3 at a position spaced from the outer peripheral part of the side surfaces of the substrate 3. Further, the reaction region group 2A is preferably formed near the center between the opposed side surfaces of the substrate 3.

Here, the plane (surface) on the optical axis of the substrate will be referred to as an optical-axis surface, and the quadrangle in the surroundings of the optical-axis surface will be referred to as side surfaces.

Incidentally, the height position in the substrate 3 at which the reaction region group 2A is formed is not particularly restricted.

The reaction microchip (substrate 3) provided with the reaction region group 2A (reaction regions 2) may be formed by use of a single or a plurality of substrates.

The method for forming the single or plurality of reaction regions 2 in the substrate 3 is not specifically restricted. Preferable examples of the forming method include wet etching or dry etching of a glass-made substrate layer, and nanoprinting, injection molding or cutting of a plastic-made substrate layer.

For example, the reaction region group 2A (the reaction regions 2) may be formed by a method in which a single or a plurality of reaction regions 2 of a desired shape are formed on a substrate by polishing-and-cutting, molding (casting) or the like and another substrate is placed on top of this substrate.

In addition, the material of the substrate 3 is not specifically restricted. Preferably, the material is appropriately selected taking into account the detection method, ease of processing, durability, etc. The material may be selected from among light-transmitting materials, appropriately according to the desired detection method. Examples of the material include glasses and various plastics (polypropylene, polycarbonate, cycloolefin polymers, polydimethylsiloxane, etc.).

The reaction regions 2 of the reaction region group 2A thus formed may be filled with reagents necessary for reactions of object substances to be detected.

### (2) Reaction Temperature Control Section

The reaction temperature control section includes a temperature control section 4 which is disposed at an outer peripheral edge part of the reaction region group 2A (hereafter referred to also as “first temperature control section 4”) and a planar temperature control section 5 (hereafter referred to also as “second temperature control section 5”). Here, a configuration is adopted in which these temperature control sections are disposed opposite to each other, with the reaction region group 2A (which has the reaction regions 2 to be reaction sites for reactions) therebetween. It suffices for these temperature control sections to be opposed to each other, with the reaction region group 2A therebetween. For example, when one of the temperature control sections is located on the upper side of the reaction region group, it suffices for the other to be located on the lower side of the reaction region group. The same applies also to the left and right sides with reference to the reaction region group. From the viewpoint of working efficiency, a structure is desired in which the temperature control section is thermally insulated from a casing of the reaction treatment device according to an embodiment of the present disclosure.

Further, a configuration is preferably adopted in which the substrate 3 having the reaction region group 2A therein is disposed, in a sandwiched manner, between the first temperature control section 4 and the second temperature control section 5. In addition, it is more desirable that the first temperature control section 4 and the second temperature control section 5 are so disposed as to make contact with the substrate 3.

In this instance, it is preferable that temperature control in the first temperature control section at least is performed by a temperature control mechanism (not shown) of the reaction temperature control section. Further, it is more preferable that temperature control in the first temperature control section 4 and the planar second temperature control section 5 is performed by the temperature control mechanism. In this case, temperature sensors, for example, thermocouple sensors, are preferably disposed. For instance, the following section (a) and (b) may be mentioned.
(a) The second temperature control section 5 is heated at a set temperature. Such heating as to compensate for the heat released (lost) is conducted by the first temperature section 4, thereby conditioning the deviation between the set temperature and a reaction temperature.
(b) The first temperature control section 4 is heated at a set temperature. The deviation between the set temperature and the reaction temperature is conditioned while heating by the second temperature control section 5.

Further, feedback control such as PID control may be adopted in the temperature control unit, whereby a higher accuracy of temperature control can be achieved.

As a result of the foregoing, the reaction region group as a whole (the reaction regions) in the substrate can be heated and cooled in a uniform manner. In other words, the temperature distribution throughout the reaction region group can be made uniform, whereby stability of reactions in the reaction regions can also be guaranteed. Specifically, temperature control of the reaction region group as a whole can be easily carried out, scattering of heat from reaction region to reaction region can be reduced, and the temperature control can be performed with high accuracy. Besides, even when many samples are simultaneously subjected to reaction treatments, the reaction treatments can be carried out in substantially the same reaction conditions, without depending on the arranging method for the reaction regions. Therefore, reaction detection accuracy and working efficiency are also enhanced. Moreover, it is unnecessary to take into special account the arranging method for the reaction regions, so that the degree of freedom in designing the reaction region group in the substrate is also enhanced.

Incidentally, a heater structure in which holes are bored in parts corresponding to the reaction regions can also be adopted. In this heater structure, care should be taken in designing, since the temperature distribution may be varied according to the design of the structures in the surroundings of the holes. In addition, a configuration in which the temperature control sections are provided in such a manner as to correspond to the individual reaction regions results in that when the number of the reaction regions is increased, processing of the heaters so as to correspond to a design of the reaction region group is necessary each time of the designing of the reaction region group, and designing of a temperature distribution is also necessary each time. Taking this point into account, the configuration of the reaction temperature control section in the first embodiment can be said to have very excellent operation and effect.

### (1.2a) First Temperature Control Section

The first temperature control section 4 is disposed at an outer peripheral edge part of the reaction region group 2A. This arrangement makes it possible to heat or cool mainly the outer peripheral edge part of the reaction region group 2A. Further, with the planar temperature control section 5 (described later) used jointly, scattering of reaction temperature among the reaction regions in the reaction region group can be reduced, and temperature control can be performed accurately.

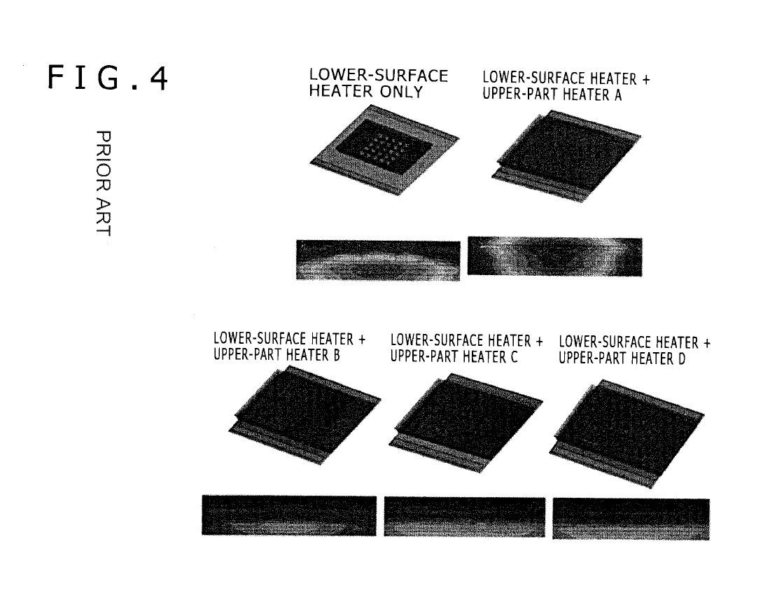

The first temperature control section 4 is preferably frame-like in shape. Further the shape of the frame body portion is preferably a shape that has an inner peripheral part 41 and an outer peripheral part 42, such as a rectangular frame-like shape or a picture frame-like shape (see FIGS. 2 and 3). Further, the frame body shape is preferably a shape that has a non-heating part near a central area (reaction region group 2A). This makes it possible to prevent local heating from occurring due to the structure in which the number of heat release paths is smaller in the central area of the reaction region group than in the peripheral area of the reaction region group. Further, a structure in which the first temperature control section 4 is hollow near its center is preferable, since it is thereby possible to easily prevent abnormal local heating in the central area (abnormal heating in the central area) which would be generated in the case where a planar heater is used alone or where a planar heater and a planar heater are combined with each other (see FIG. 4).

Incidentally, the frame body portion is preferably set substantially equal to the substrate 3 in breadth and depth.

In addition, as shown in FIG. 3, the first temperature control section 4 is provided with a single or a plurality of cutouts 43 in an inner peripheral part 41 and/or an outer peripheral part 42 of the frame body portion thereof. The shape of the cutout is not specifically restricted, insofar as it is a shape obtained by, or as if by, cutting away a part of the frame body portion. Examples of the shape of the cutout include polygonal shapes (rectangle, square, hexagon, etc.) and semi-elliptic shapes (semi-circular shape, etc.).

The cutout or cutouts 43 are each preferably provided at a place where local heating is liable to occur due to concentration of heat of the first temperature control section 4. This ensures that the reaction region group 2A as a whole can be temperature-controlled substantially uniformly, with higher accuracy (see FIG. 4). In this instance, the provision of the cutout or cutouts 43 is preferably made according to the shape of the substrate 3, from the viewpoint of reduction in cost and enhancement of working efficiency. Furthermore, a plurality of the cutouts 43 are preferably provided so as to be opposed to each other.

In addition, a single or a plurality of the cutouts 43 are preferably provided in a central part of each edge of the inner peripheral part 41 of the body frame portion. Further, a single or a plurality of the cutouts 43 are preferably provided in the corners of the outer peripheral part 42. The arrangement of these cutouts 43 may be appropriately combined.

For example, a frame-shaped first temperature control section 4a may be mentioned in which two cutouts 43 are provided respectively in central parts of opposite edges of the inner peripheral part 41. In addition, a frame-shaped first temperature control section 4b may be mentioned in which four cutouts 43 are provided respectively in the four corners of the outer peripheral part 42. Besides, a frame-shaped first temperature control section 4c may be mentioned in which four cutouts 43 are provided respectively in central parts of two pairs of opposed edges of the inner peripheral parts 41 and four cutouts 43 are provided respectively in the four corners of the outer peripheral part 42. Incidentally, these examples are not restrictive.

The first temperature control section 4 is preferably so disposed as to make contact with a surface of the substrate 3 (preferably, an outer peripheral edge part of the reaction region group 2A). In this instance, a highly heat-conductive member may be interposed between these two members. This promises accurate temperature control. Incidentally, examples of the surface of the substrate in this case include the optical-axis surface, the upper surface, the lower surface, the left surface, and the right surface.

In addition, the temperature control mechanism of the first temperature control section 4 is not specifically restricted. Examples of the mechanism include heaters such as ceramic ones, heating wire, Peltier element, etc., and transparent conductive films such as light-transmitting ITO heater. Besides, since the temperature control section 4 in the present disclosure adopts a frame-like shape, heat release efficiency is high in the central area of the reaction region group 2A, even a temperature control mechanism lacking a cooling mechanism, such as a ceramic heater or heating wire heater, may be used favorably.

Further, where the reaction treatment device in the present disclosure is an optical detection device (nucleic acid amplification reaction device or the like), the configuration in which the temperature control section 4 is disposed at the outer peripheral edge part of the reaction region group ensures that the temperature control mechanism may be assembled by use of a light-screening member, in other words, a low-transmittance member or non-light-transmitting member. Thus, the configuration in which the temperature control section is frame-shaped is advantageous from the viewpoint of cost reduction and facilitated measurement.

### (1.2b) Second Temperature Control Section

The second temperature control section 5 is disposed in a planar form. With this temperature control section formed to have a planar shape (flat plate section), it is possible to heat or cool mainly the vicinity of the reaction region group 2A. Further, using this together with the above-mentioned first temperature control section 4, scattering of reaction temperature among the reaction regions in the reaction region group can be reduced, and temperature control can be performed with high accuracy.

It suffices for the second temperature control section 5 to be planar in shape and be capable of controlling the reaction temperature in the reaction region group 2A; therefore, for example, a thin membrane shape, a flat plate-like shape and the like can also be adopted, other than the planar-shape (see FIGS. 1 and 2). In this instance, the area of temperature control is preferably set to be mainly the reaction region group 2A as a whole. Although the temperature control section 5 may have a plurality of temperature control mechanisms for temperature control on an areal basis, the temperature control section 5 having only a single temperature control mechanism is preferable from the viewpoint of cost reduction and ease of temperature control. Even with such a single temperature control mechanism, its cooperation with the above-mentioned first temperature control section 4 ensures that reaction temperature in the reaction region group can be controlled accurately.

The second temperature control section 5 is preferably so disposed as to make contact with a surface of the substrate 3 (preferably, at least the area of the reaction region group 2A). In this instance, a highly heat-conductive member may be interposed between the two members. This enables accurate temperature control. Incidentally, examples of the surface of the substrate in this case include the optical-axis surface, the upper surface, the lower surface, the left surface, and the right surface.

It should be understood that various changes and modifications to the presently preferred embodiments described herein will be apparent to those skilled in the art. Such changes and modifications can be made without departing from the spirit and scope and without diminishing its intended advantages. It is therefore intended that such changes and modifications be covered by the appended claims.

Incidentally, the configuration in which the first temperature control section 4 is disposed at the outer peripheral edge part of the reaction region group 2A and makes surface contact with the substrate 3 and the planar form (planar part) of the second temperature control section 5 makes surface contact with the substrate is preferable because it ensures that temperature control can be carried out easily and with high accuracy. With such a configuration adopted, the number of component parts can be reduced, and electric wiring can be laid around easily. By making the most of these merits, it is possible to design the reaction section in a compact form. Since the heat generating sections can be reduced in size, the total heat capacity can be reduced, and a large saving of electric power can be achieved.

Examples of a temperature control mechanism for the planar second temperature control section 5 include the one which has been described in connection with the first temperature control section 4 above. In addition, a high-light-transmittance member, for example, a transparent conductive film, is preferably used. On the other hand, if a mechanism for reflecting the light generated from the reaction regions 2 is adopted, favorable measurement can be achieved even when a highly light-screening member, for example, a ceramic heater or a heating wire heater is used.

### (3) Irradiation Section

It suffices for the irradiation section 8 to have a light source (not shown) and be so configured that the reaction regions 2 are irradiated with the light L outputted from the light source. Specifically, it suffices that the irradiation section 8 can irradiate the upper surface, the lower surface or the like of the reaction regions 2 (substrate 3) with the light L going out from the light source, for detection of nucleic acid amplification (products) generated attendant on the progress of nucleic acid amplification reaction. For instance, the light source may be disposed on the upper side or the lower side of the reaction regions 2, or a light guide member (not shown) for guiding the light L outputted from the light source to the reaction regions 2 may be disposed.

Among these choices, it is preferable that the irradiation section 8 is provided with a light guide member by which the light generated from the light source is guided to the reaction regions 2. The light guide member is provided with a light incidence end section, and the light outputted from a single or a plurality of light sources is incident on the light incidence end section. A member (e.g., prism, reflector, rugged pattern, or the like) by which the incident light is guided to each of the reaction regions is provided inside the light guide member.

With the light guide member disposed, the number of light sources can be reduced, and a single or a plurality of reaction regions 2 on the substrate 3 can be irradiated with light uniformly. Therefore, detection sensitivity and detection accuracy in turbidity detection are favorable. Moreover, since the number of the light sources can be reduced, the reaction treatment device as a whole can be reduced in size, particularly in thickness, and a reduction in electric power consumption can be achieved.

The light source is not specifically restricted, but is preferably one capable of emitting desired light which enables favorable detection of an object nucleic acid amplification product. Examples of the light source include a laser light source, a white-color or monochromatic light emitting diode (LED), a mercury vapor lamp, and a tungsten lamp. In addition, the LED is advantageous, since desired light components can also be obtained through use of various filters.

Incidentally, the laser light source is not specifically restricted by the kind of laser light. It suffices for the laser light source to be one that emits argon ion (Ar) laser, helium-neon (He—Ne) laser, dye laser, krypton (Kr) laser or the like. These laser light sources may be used either singly or in arbitrary combination of two or more of them.

Incidentally, as shown in FIG. 1, the light L from the irradiation section 8 reaches the reaction region 2, to be turned into light L3 by a nucleic acid amplification product produced attendant on the progress of a reaction in the reaction region 2. Then, the light L3 (quantity of forward, back, or side-way scattered light, quantity of transmitted light, quantity of fluorescent light, or the like) generated from the nucleic acid amplification product is appropriately transmitted through a diaphragm, a condenser lens, a fluorescence filter or the like appropriately, before being detected by the detection section 9 (optical detector).

### (4) Detection Section

The detection section 9 may be any one that can detect the quantity of light going out from the other end (specifically, the bottom surface) of the reaction region 2. The detection section 9 is provided at least with an optical detector.

The optical detector is not particularly restricted. Examples of the optical detector include area imaging elements such as a photodiode (PD) array, a CCD image sensor, a CMOS image sensor, etc., small-type sensors, line scan sensors, and PMT (photomultiplier tubes), which may be used in appropriate combination. By the optical detector, the nucleic acid amplification product or the like is detected.

Incidentally, in the nucleic acid amplification reaction device in the present disclosure, the excitation filter 6 and the fluorescence filter 7 may be appropriately disposed, extended or removed. The excitation filter makes it possible to obtain a light component of a desired specific wavelength, or remove unnecessary light component, according to the detection method for the nucleic acid amplification reaction. In addition, the fluorescence filter makes it possible to obtain a light component (scattered light, transmitted light, or fluorescent light) necessary for detection. Consequently, detection sensitivity and detection accuracy are enhanced.

### <2. Nucleic Acid Amplification Reaction Device>

The reaction treatment device 1 in the present disclosure is high in the degree of freedom in design, since in the case of the same type of microchip, temperature section is not changed depending on the number of wells, the number of microwells or the arrangement method. In addition, the microchip can maintain temperature uniformity notwithstanding the heat capacity is small. Specifically, in a microchip in which microwells are arranged in parallel, the microchip designed for optical detection of the reaction states in the reaction sites can be heated uniformly. The reaction treatment device 1 in the present disclosure thus has little scattering of reaction; therefore, it is advantageous to use the reaction treatment device 1 as a nucleic acid amplification reaction device which is desired to have a high detection accuracy.

Now, the nucleic acid amplification reaction will be described below.

### (1) Nucleic Acid Amplification Reaction

In the description of the present disclosure, the term “nucleic acid amplification reaction” includes not only the ordinary PCR (polymerase chain reaction) method in which a temperature cycle is used but also various isothermal amplification methods which do not involve a temperature cycle. Examples of the isothermal amplification method include LAMP (Loop-Mediated Isothermal Amplification) method, SMAP (Smart Amplification Process) method, NASBA (Nucleic Acid Sequence-Based Amplification) method, ICAN (Isothermal and Chimeric primer-initiated Amplification of Nucleic acids) method (registered trademark), TRC (transcription-reverse transcription concerted) method, SDA (strand displacement amplification) method, TMA (transcription-mediated amplification) method, and RCA (rolling circle amplification) method.

Other than the above-mentioned, the term “nucleic acid amplification reaction” widely includes temperature-varied or isothermal nucleic acid amplification reactions aiming at amplification of nucleic acid. Besides, these nucleic acid amplification reactions include reactions involving determination of amplified nucleic acid chains, such as real-time PCR (RT-PCR) method and RT-LAMP method.

In addition, the term “reagents” section the reagents necessary for obtaining amplified nucleic acid chains in the above-mentioned nucleic acid amplification reactions. Specific examples of the reagent include oligonucleotide primers set to a base sequence complementary to the target nucleic acid chain, a nucleic acid monomer (dNTP), enzymes, and buffer solutes.

In the PCR method, an amplification cycle of “thermal denaturation (about 95° C.)→annealing of primer (about 55 to 60° C.)→elongation reaction (about 72° C.)” is conducted successively.

In addition, the LAMP method is a method in which dsDNA is obtained as an amplification product from DNA or RNA at a fixed temperature by utilizing loop formation of DNA. In an example, the components (i), (ii), and (iii) set forth below are added, and incubation is conducted at such a temperature that the inner primer can form a stable base pair bond with a complementary sequence on a template nucleic acid and that a chain substitution type polymerase can keep enzyme activity, resulting in progress of the desired reaction. The incubation temperature in this instance is preferably 50 to 70° C., and the time is preferably about 1 minute to 10 hours.

Component (i): two kinds of inner primers, or plus two kinds of outer primers, or plus two kinds of loop primers;

Component (ii): chain substitution type polymerase;

Component (III): substrate nucleotide.

### (2) Detection Method for Nucleic Acid Amplification (Products)

Examples of the detection method for the nucleic acid amplification include methods in which a turbid substance, a fluorescent substance, a chemiluminescent substance or the like is used.

Besides, examples of the method using a turbid substance include a method using a precipitated substance arising from pyrophosphoric acid, which is produced as a result of the nucleic acid amplification reaction, and a metal ion capable of bonding thereto. The metal ion is a univalent or bivalent metal ion which, upon bonding to pyrophosphoric acid, forms a salt insoluble or difficultly soluble in water, to be a turbid substance.

Specific examples of the metal ion include alkali metal ions, alkaline earth metal ions and bivalent transition metal ions. Among these metal ions, preferred are one or at least two selected from among alkaline earth metal ions such as magnesium(II), calcium(II) and barium(II) ions, and bivalent transition metal ions such as zinc(II), lead(II), manganese(II), nickel(II) and iron(II) ions, etc. More preferred are magnesium(II), manganese(II), nickel(II) and iron(II) ions.

The concentration of the metal ions to be added is preferably 0.01 to 100 mM. The detection wavelength is preferably 300 to 800 nm.

In addition, examples of the method in which a fluorescent substance or a chemiluminescent substance is used include an intercalation method using a fluorescent dye (derivative) generating fluorescent light by being inserted (intercalated) specifically into a double strand nucleic acid, and a labeled probe method using a probe prepared by bonding a fluorescent dye to an oligonucleotide specific to a nucleic acid sequence to be amplified.

Examples of the labeled probe method include a hybridization (Hyb) probe method and a hydrolysis (TaqMan) probe method.

The Hyb probe method is a method using two kinds of probes consisting of a probe labeled with a donor dye and a probe labeled with an acceptor dye, so designed that the two kinds of probes come into proximity to each other. When the two kinds of probes are hybridized with a target nucleic acid, the acceptor dye excited by the donor dye generates fluorescent light.

On the other hand, the TaqMan probe method is a method using a probe so labeled that a reporter dye and a quencher dye come into proximity to each other. At the time of elongation of a nucleic acid, the probe is hydrolyzed, whereon the quencher dye and the reporter dye are separated away from each other, and the reporter dye generates fluorescent light when excited.

Examples of the fluorescent dye (derivative) to be used in the method using a fluorescent substance include SYBR (registered trademark) Green I, SYBR (registered trademark) Green II, SYBR (registered trademark) Gold, YO (Oxazole Yellow), TO (Thiazole Orange), PG (Pico (registered trademark) Green), and ethidium bromide.

Examples of the organic compound to be used in the method using a chemiluminescent substance include luminal, lophine, lucigenin, and oxalic esters.

### <3. Operation of Reaction Treatment Device 1 of First Embodiment>

Now, operation of the above-mentioned reaction treatment device 1 will be described below.

Description will be made of a configuration in which the reaction region group 2A formed by disposing the reaction regions 2 to be reaction sites for various reactions is interposed, in a sandwiched manner, between the first temperature control section 4 and the second temperature control section 5. The substrate temperature at the outer peripheral edge part of the reaction region group 2A is controlled by the first temperature control section 4, and the substrate temperature of the substrate 3 (at least the reaction region group 2A) is controlled by the second temperature control section 5. By this cooperation, the reaction temperature in the reaction regions 2 in the reaction region group 2A is controlled. Then, the reactions in the reaction regions 2 are controlled while heating or cooling the substrate 3, as required, by such control as feedback control.

Thus, with one of the temperature control sections disposed at the outer peripheral edge part of the reaction region group, easy heat release is permitted in a central portion of the reaction region group, whereby concentrated heating can be prevented from occurring in this portion. Moreover, by cooperation of this temperature control section with the second temperature control section 5, it is also made possible to make uniform the temperature distribution in the reaction region group as a whole.

This ensures that the temperature control can be made easily and with high accuracy, and reaction products in the reaction regions can be thereby provided stably. Moreover, it becomes possible to design the reaction section in a compact form, as above-mentioned. In addition, since the heat generating section can be reduced in size, the overall heat capacity can be reduced, and a large saving of electric power can be achieved.

Further, preferably, a single or a plurality of cutouts 43 are provided in the outer peripheral part 42 and/or the inner peripheral part 41 of the frame body portion of the first temperature control section 4, thereby restraining local heating more assuredly. With the cutouts provided at parts where local heating is liable to occur through concentration of heat, the temperature control can be performed more accurately and more easily.

Incidentally, where the reactions in the reaction regions 2 are nucleic acid amplification reactions, it suffices to conduct temperature control according to the above-mentioned nucleic acid amplification reaction.

### <4. Operation of Nucleic Acid Amplification Reaction Device Utilizing Reaction Treatment Device 1>

Now, operation of the above-mentioned nucleic acid amplification reaction device will be described below.

Incidentally, the temperature control in the nucleic acid amplification reaction is as above-mentioned, and, therefore, description thereof is omitted here. In addition, detection of the nucleic acid amplification may be carried out on a real-time basis while performing the temperature control.

Light L1 is emitted from the light source 8, and it is turned into light L2 by the excitation filter 6. The light L2 is radiated to one end (upper surface) of the reaction region 2 to be a reaction site for a nucleic acid amplification reaction, to be incident on the well. In this instance, light L3 (fluorescent light, scattered light, transmitted light, or the like) generated due to the nucleic acid amplification product is generated from the reaction region 2, and is let go out from the other end (bottom surface) of the reaction region 2. The light L3 is turned into a desired light component L4 (e.g., a specified fluorescent light component, scattered light component, or transmitted light component or the like) by the fluorescence filter 7. The light L4 is fed to the detection section 9 (optical detector), which detects the quantity of the light let go out, whereby the nucleic acid amplification product produced attendant on the progress of the amplification reaction can be measured.

Incidentally, in the case of turbidity detection, the excitation filter 6 and the fluorescence filter 7 may be omitted appropriately.

### (1) Modification

The above-described nucleic acid amplification reaction device according to an embodiment of the present disclosure can be used as a LAMP device or a PCR device, and can be used to determine nucleic acid by fluorescent substance detection or turbid substance detection. While turbid substance detection will be shown below, fluorescent substance detection can also be conducted according to the fluorescence detection method as above-mentioned.

### (4.1a) Operation of RT-LAMP Device

Now, a nucleic acid detection method according to the procedure of step S11 in using an RT-LAMP device will be described below.

In a temperature control step (step S11), a setting is made such that a fixed temperature (60 to 65° C.) is established in the reaction regions 2, whereby the nucleic acid in each reaction region 2 is gradually amplified. Incidentally, in this LAMP method, thermal denaturation from single strand to double strand is unnecessary, and annealing of primer and elongation of nucleic acid are repeated under isothermal conditions.

As a result of the nucleic acid amplification reaction, pyrophosphoric acid is produced, a metal ion is bonded to the pyrophosphoric acid to form an insoluble or difficultly soluble salt, which becomes a turbid substance (measurement wavelength: 300 to 800 nm). Upon irradiation of the turbid substance with incident light (light L), scattered light is generated. The quantity of the scattered light is measured on a real-time basis by the detection section 9, and the measurement is put to quantification. Besides, quantification from the quantity of transmitted light is also possible.

### (4.1b) Operation of RT-PCR Device

Now, a nucleic acid detection method according to step Sp1 (thermal denaturation), step Sp2 (annealing of primer) and step Sp3 (elongation of DNA) in using an RT-PCR device will be described below.

In the thermal denaturation step (step Sp1), the temperature in the reaction regions 2 is controlled to 95° C. by the temperature control section, whereby double-stranded DNA is denatured into single-stranded DNA.

In the subsequent annealing step (step Sp2), the temperature in the reaction regions 2 is set to 55° C., whereby the primer is bonded to a base sequence complementary to the single-stranded DNA.

In the next DNA elongation step (step Sp3), the temperature in the reaction regions 2 is controlled to 72° C., whereby a polymerase reaction is made to proceed, with the primer as a starting point of DNA synthesis, thereby effecting elongation of cDNA.

With such a temperature cycle of steps Sp1 to Sp3 repeated, the DNA in each reaction region 2 is gradually amplified. As a result of the nucleic acid amplification reaction, pyrophosphoric acid is produced, then the turbid substance is detected in the above-mentioned manner, and the quantity of the nucleic acid is put to quantification.

### <5. Reaction Treatment Device of Second Embodiment>

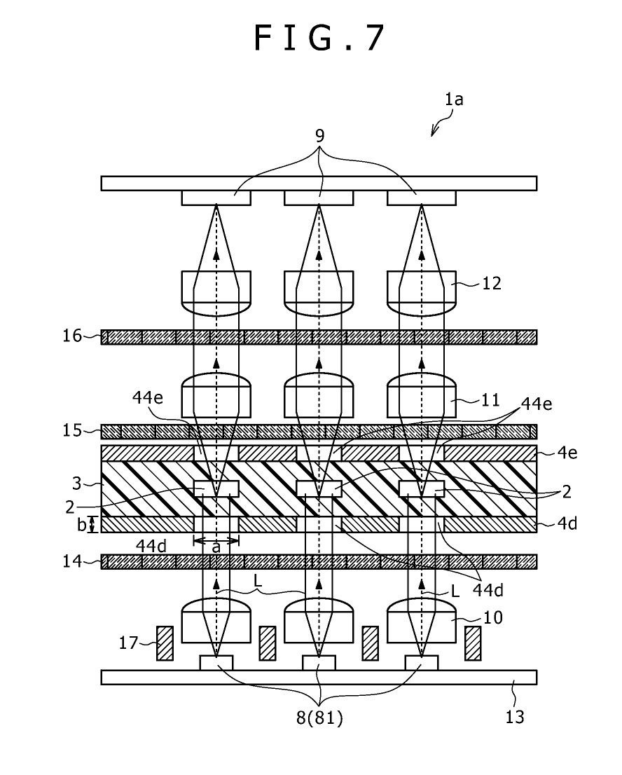

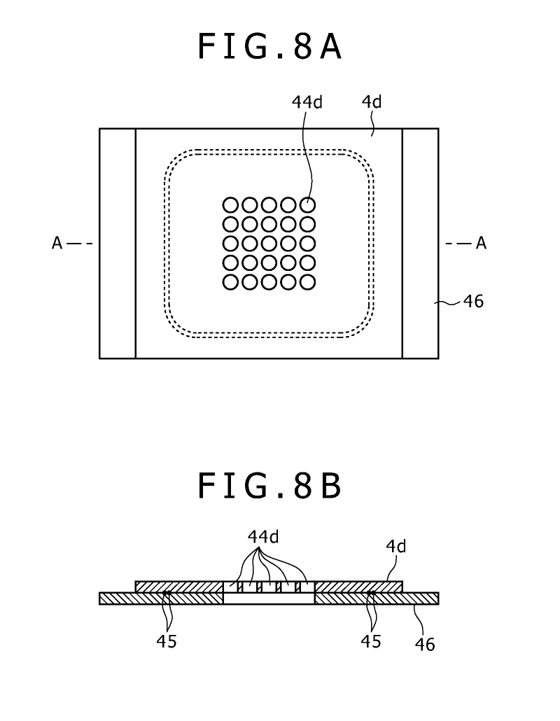

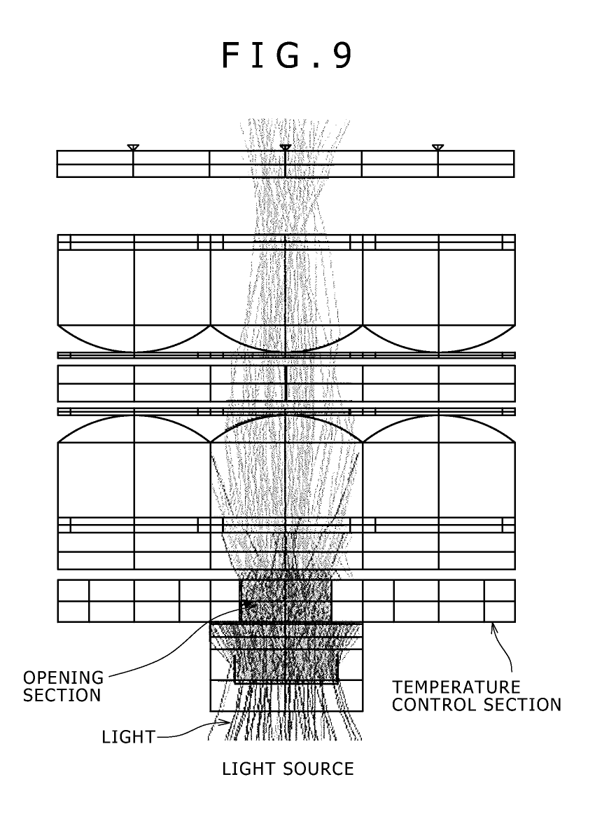

FIG. 7 is a conceptual diagram of a reaction treatment device 1 a according to a second embodiment of the present disclosure. FIG. 8A is a top view of a temperature control section 4d according to the second embodiment, and FIG. 8B is a sectional view taken along line A-A of FIG. 8A. FIG. 9 is a conceptual diagram illustrating propagation of rays of light from a light source in the case where the temperature control section 4d according to the second embodiment is used.

As shown in FIG. 7, the reaction treatment device la of the second embodiment includes at least a temperature control section (4d or 4e) which controls the temperature of an outer peripheral edge part of a reaction region group 2A.

Further, the reaction treatment device 1 a according to the second embodiment of the present disclosure can be used also as an optical detection device or a nucleic acid amplification reaction device. For instance, as shown in FIG. 7, it is desirable that the reaction treatment device 1 a further includes irradiation sections 8 (light sources 81), an excitation filter 14, detection filters 15, 16, diaphragms 17, and detection sections 9. As a specific example, there may be mentioned a configuration wherein at least the irradiation sections 8 (light sources 81) which irradiate the reaction regions 2 with light and the detection sections 9 which detect light coming from the reaction regions 2 are provided. Incidentally, the irradiation sections 8 (light sources 81) are disposed on a support 13.

In addition, though not shown in the drawing, a configuration may be adopted wherein, for example, detection sections 9 are disposed on the side of the irradiation sections 8 (light sources 81) and the light (scattered light or fluorescent light or the like) generated from the reaction regions 2 is reflected toward the detection sections 9 so that the light can be detected by the detection sections 9.

Besides, it is preferable that control sections (not shown) are provided for controlling various kinds of operations (e.g., light control, temperature control, nucleic acid amplification reaction, detection control, computation of quantity of light detected, monitoring, etc.).

Now, the temperature control section 4d in the reaction treatment device (nucleic acid amplification reaction device) 1a in the second embodiment of the present disclosure will be described in detail below. Incidentally, the reaction region group 2A (reaction regions 2), the substrate 3, the irradiation section 8 (light source 81), the detection section 9 are the same as described in the first embodiment above, and descriptions of them will therefore be omitted.

### (1) Temperature Control Section

The temperature control section 4d has a flat plate-like shape so sized as to overlap with the outer peripheral edge part of the reaction region group 2A. In addition, in the plane of the flat plate-like shape, the temperature control section 4d is provided with light-transmitting opening sections 44d at positions corresponding to the reaction regions 2 in the reaction region group 2A. This configuration makes it possible to heat or cool mainly the outer peripheral edge part and also to heat or cool the vicinity of the opening sections 44d. Further, the opening sections 44d serves as heat release paths, whereby local heating in the area of the reaction region group 2A can be prevented.

In addition, the temperature control section 4d having the opening section 44d is preferably disposed so as to make contact with the substrate 3. This configuration enables efficient heating or cooling of the reaction region group 2A disposed in the substrate 3. From the viewpoint of the efficient temperature control, it is preferable that two such temperature control sections having the opening section are provided and that the temperature control section 4d and the temperature control section 4e are disposed opposite to each other, with the reaction region group 2A (substrate 3) therebetween (see FIG. 7).

Incidentally, as the temperature control mechanism of the temperature control section 4d having the opening section 44d, one which is the same as or similar to the temperature control mechanism described in the first embodiment above can be used. In FIG. 8 a temperature control section 4d obtained by winding a heater wire around a metallic plate is shown as an example. Examples of the metal of the metallic plate include aluminum, stainless steel, copper, and nickel (Ni). The metallic plate is preferably formed with a groove or projection so that the heater wire 45 can be easily wound around the metallic plate.

The shape of the opening section 44d is not particularly restricted, and is preferably a shape corresponding to the shape of each reaction region 2. The shape is not restricted to circle but may be square or polygon, insofar as it corresponds to each reaction region 2. The surface of the shape of the opening section 44d is preferably provided substantially in parallel to the reaction region 2.

Examples of the three-dimensional shape of the opening section 44d include a cylindrical shape, prismatic shapes, and polyhedral shapes. For example, a shape provided therein with a taper may be adopted.

It is preferable, from the viewpoint of cost, that the opening section 44d has a single or a plurality of portions (holes or the like) penetrating the light-screening body which are formed in a region corresponding to each reaction region 2.

The temperature control section 4d having the opening section 44d can be produced by providing the above-mentioned metallic plate with a single or a plurality of opening sections in a predetermined pattern by such a technique as, for example, blanking, cutting, and photo-etching.

The temperature control section 4d having the opening section 44d is preferably provided with a heat insulating section 46 for restraining release of heat from the temperature control section 4d, as shown in FIG. 8. It is also preferable that the temperature control section 4d is disposed between the substrate 3 and the heat insulating section 46. The heat insulating section 46 is preferably disposed in contact with the temperature control section 4d having the opening section 44d. In addition, the heat insulating section 46 is preferably disposed on the side (the heat irradiation section 8 side) opposite to the substrate 3 side with reference to the temperature control section 4d. Such a heat insulating section 46 ensures that the heat from the temperature control section 4d having the opening section 44d can be efficiently conducted to the substrate 3 (reaction region group 2A) side.

Of the heat insulating section 46, the portion corresponding to the whole area of the reaction region group 2A may be opened, as shown in FIG. 8B, which is preferable in that light can easily pass to each of the reaction regions 2. Besides, though not shown in the drawings, the heat insulating section 46 may be perforated at positions corresponding respectively to the reaction regions 2 in the reaction region group 2A. Further, though not shown, the heat insulating section may be provided also in the area corresponding to the whole area of the reaction region group 2A, which is preferable in that the heat from the temperature control section 4d is more unlikely to be released. In this case, it is preferable to use a light-transmitting, transparent member in the area corresponding to the whole area of the reaction region group 2A.

The heat insulating section 46 may be united with the temperature control section 4d having the opening section 44d, to form a temperature control unit which includes the temperature control section 4d and the heat insulating section 46. This makes it possible to reduce the number of component parts and to simplify the assembly process, in assembling the device according to the embodiment of the present disclosure.

Examples of the material for the heat insulating section 46 include synthetic resins such as polycarbonate, polyethylene terephthalate, poly(meth)acrylate, polyurethane, polystyrene, etc. and their foams.

The temperature control section 4d having the opening section 44d is preferably a light-screening body. With the temperature control section 4d formed as a light-screening body, light is permitted to pass only through the opening sections 44d, and passage of light through the other portion than the opening sections 44d can be prevented (see FIG. 9). Therefore, when the temperature control section 4d is disposed on the irradiation section 8 side, the direction in which the light from the light source 81 in the irradiation section 8 is incident on each reaction region 2 can be restricted. In addition, when the temperature control section 4e is disposed on the detection section 9 side, the direction in which light goes out from each reaction region 2 can be restricted. Such an arrangement of the temperature control section(s) ensures that stray light (crosstalk) from the surrounding reaction regions (particularly, the adjacent reaction regions) which would cause detections errors can be restrained, whereby detection accuracy is enhanced (see FIG. 9). From the viewpoint of enhancing the detection accuracy, it is preferable that two such temperature control sections are provided and that the temperature control section 4d and the temperature control section 4e are disposed opposite to each other, with the reaction region group 2A (substrate 3) therebetween (see FIG. 7). The two temperature control sections 4d and 4e are preferably identical, from the viewpoint of cost.

In addition, the temperature control sections 4d and 4e having the opening sections are preferably disposed in contact with the surface of the substrate 3. This ensures that intrusion of stray light from the surrounding reaction regions can be reduced more favorably. Incidentally, the temperature control section 4e can also be provided with the above-mentioned heat insulating section. In this case, the temperature control section 4e having the opening sections 44e is preferably disposed between the substrate and the heat insulating section. The heat insulating section is preferably disposed on the side (the detection section 9 side) opposite to the substrate 3 side with reference to the temperature control section 4e having the opening sections 44e.

The opening sections 44d and 44e preferably have a predetermined depth (thickness), in order to restrict the outgoing (emission) direction and incidence direction of light. By controlling the depth, it is possible to restrict the outgoing direction of light from within the reaction region 2 and the incidence direction of light from the irradiation section 8 (light source 81). From this point of view, the thickness b of the opening section (the thickness of the temperature control section) is preferably in the range of 0.2 to 1.5 mm, more preferably 0.5 to 1.0 mm. Besides, by for example regulating the width a (horizontal edges or diameter) of the inside of the opening section or the thickness b of the opening section, it is also possible to control the angle of incidence of light on each reaction region 2 and the angle of incidence of light on the detection filter 15. Since it is possible to control the incidence angle of light in this way, it is also possible to adapt to various detection filters by controlling the width a of the inside of the opening section and/or the thickness b of the opening section. As the width a of the inside of the opening section set smaller and the thickness b of the opening section set larger, stray light can be restrained more satisfactorily.

Hitherto, in order to avoid mis-detection due to stray light (crosstalk), excitation/light detection has been applied to the individual reaction regions on a time division basis. Accordingly, it has been necessary to arrange one light source and one detector for each reaction region. In addition, since the time required for one cycle of detection is proportional to the number of the reaction regions, there has been a difficulty in terms of throughput in the case of measuring a large number of specimens, for example, in the case of using a 96-holed plate or the like.

When the temperature control section(s) having the opening sections is adopted, however, stay light from the surrounding reaction regions can be suppressed. In addition, it is also made possible to carry out in a stroke the excitation/light detection, which has been conducted on a time division basis in the past. Further, adoption of the light-transmitting member makes it possible to perform excitation in a stroke on an areal basis, to perform detection with uniform light, and to greatly shorten the detection time required for a large number of reaction regions.

In the reaction treatment device 1 a according to the second embodiment as above-described, the temperature control section 4d having the opening sections 44d can be replaced by the first temperature control section or the second temperature control section described in the first embodiment above. Besides, in the reaction treatment device 1a according to the second embodiment, the temperature control section 4e having the opening sections 44e can be replaced by the first temperature control section or the second temperature control section described in the first embodiment.

Incidentally, it is advantageous for the reaction treatment device 1a of the second embodiment to be used as a nucleic acid amplification reaction device, for the same reason as described above in relation to the reaction treatment device 1 of the first embodiment.

### <6. Operation of Reaction Treatment Device 1a of Second Embodiment>

Now, operation of the above-described reaction treatment device la will be described below.

[The Case Where One Temperature Control Section (4d or 4e) is Provided]

First, description will be made of a configuration wherein a temperature control section (4d or 4e) is disposed on the irradiation section 8 side or the detection section 9 side of the substrate 3 provided therein with the reaction regions 2 to be reaction sites for various reactions. The substrate temperature at the outer peripheral edge part of the reaction region group 2A is mainly controlled by the temperature control section (4d or 4e), from the irradiation section 8 side or the detection section 9 side. Simultaneously, the substrate temperature in the surroundings of the opening sections (44d or 44e) (in the surroundings of each reaction region 2) is controlled from the irradiation section 8 side or the detection section 9 side. By this, the reaction temperature in the reaction regions 2 present in the reaction region group 2A is controlled. Besides, the reaction in the reaction regions 2 is controlled while heating or cooling the substrate 3, as required, by such control as feedback control.

Thus, the temperature control section (4d or 4e) is disposed at least at the outer peripheral edge part of the reaction region group 2A and the opening section (44d or 44e) is provided at the part corresponding to each reaction region 2, whereby heat is easily released from the reaction region group 2A. Accordingly, local heating in the area of the reaction region group 2A can be prevented, and the temperature distribution in each reaction region 2 can be made uniform.

[The Case Where Two Temperature Control Sections (4d and 4e) are Provided]

Description will be made of a configuration in which the substrate 3 is sandwiched or interposed between two temperature control sections 4d and 4e (see FIG. 7). The substrate temperature at the outer peripheral edge part of the reaction region group 2A is mainly controlled by the temperature control sections 4d and 4e from the irradiation section 8 side and the detection section 9 side. Simultaneously, the substrate temperature in the surroundings of the opening sections 44d and 44e of the temperature control sections 4d and 4e (in the surroundings of each reaction region 2) is also controlled from the irradiation section 8 side and the detection section 9 side. By this, the reaction temperature in the reaction regions 2 present in the reaction region group 2A is controlled. Besides, the reaction in the reaction regions 2 is controlled while heating or cooling the substrate 3, as required, by such control as feedback control.

Thus, the temperature control sections 4d and 4e are disposed at least at the outer peripheral edge part of the reaction region group 2A and the opening sections 44d and 44e are provided at the position corresponding to each reaction region 2, whereby heat is easily released from the reaction region group 2A. Therefore, local heating in the area of the reaction region group 2A can be prevented, and the temperature distribution in each reaction region 2 can be made uniform. With the substrate 3 sandwiched or interposed between the two temperature control sections 4d and 4e, temperature control with higher accuracy can be accomplished, as compared with the case of only one temperature control section (4d or 4e).

Consequently, temperature control can be performed easily and highly accurately, and, also, reaction products in the reaction regions can be provided stably. Moreover, a compact design of the reaction section can be realized, as above-mentioned. Besides, since the heat generating sections can be reduced in size owing to the compact design of the reaction section, the total heat capacity can be reduced and a large saving of electric power can be achieved.

Incidentally, where the reaction in the reaction regions 2 is a nucleic acid amplification reaction, it suffices to perform temperature control in the manner according to the case of the above-described nucleic acid amplification reaction.

### <7. Operation of Nucleic Acid Amplification Reaction Device Utilizing Reaction Treatment Device 1a of Second Embodiment>

Now, operation of the above-described nucleic acid amplification reaction device will be described below.

Incidentally, the temperature control in the nucleic acid amplification reaction is the same as above-described and, hence, description thereof is omitted here. In addition, nucleic acid amplification may be detected on a real-time basis while performing temperature control.

Light L from the light source 81 in the irradiation section 8 is radiated to the reaction region 2 containing the specimen. In this instance, each reaction region 2 may be irradiated with the light L by use of a light guide member. The excited light L passes through the opening section 44d of the temperature control section 4d, to be radiated onto each reaction region 2. In this way, the incidence direction of the light L is restricted by the passage through each opening section 44d present in the temperature control section 4d.

The light component (fluorescent light, transmitted light, scattered light, or the like) L going out from within each reaction region 2 passes through each opening 44e in the temperature control section 4e. Thus, the outgoing direction of the light component L is restricted by passage through each opening section 44e in the temperature control section 4e. This makes it possible to suppress stray light (crosstalk) from the surrounding reaction regions (particularly, the adjacent reaction regions) which would cause detection errors. Then, the light component L restricted in outgoing direction is transmitted through a detection filter 15, a condenser lens 11, a detection filter 16 and a condenser lens 12, to be a desired light component L. This light component L is detected by an optical detector in the detection section 9. In this case, since the stray light from the surrounding reaction region is suppressed, detection accuracy for the specimen in each reaction region is enhanced. With the reaction region 2 thus used as a reaction site at the time of measurement, real-time detection can be achieved, and reaction and detection can be performed in a continuous manner, which is highly convenient.

Incidentally, where the light source is a laser light source, the excitation filter may not necessarily be used, and excited light therefrom is radiated to the reaction region 2. Where the light source is an LED or the like, excited light having been transmitted through the excitation filter 14 is incident on the reaction region 2.

In addition, the excitation filter may be a multiband-pass filter, and the use of the multiband-pass filter permits a plurality of excited light components to be incident on the reaction region 2. In this case, it suffices to appropriately use as the detection filter a multiband-pass filter corresponding to the above-mentioned one. This enables a plurality of light analyses, and permits the light detection to be performed on a time division basis.

Besides, the numbers and kinds of the excitation filter(s), detection filter(s) and condenser lens(es) may be appropriately selected, as required, and they are not limited to the above-mentioned.

Incidentally, the nucleic acid amplification reaction device utilizing the reaction treatment device 1a can also be used as a LAMP device or a PCR device, and determination of nucleic acid can be performed through fluorescent substance detection or turbid substance detection.

Incidentally, embodiments of the present disclosure can assume the following configurations.

(1) A reaction treatment device including a temperature control section which controls temperature of an outer peripheral edge part of a group of reaction regions.

(2) The reaction treatment device as described in the above paragraph (1), wherein the temperature control section is a first temperature control section disposed at the outer peripheral edge part of the group of reaction regions, the reaction treatment device includes a reaction temperature control section including the first temperature control section and a planar second temperature control section, and the first temperature control section and the second temperature control section are disposed opposite to each other, with the reaction region group therebetween.

(3) The reaction treatment device as described in the above paragraph (2), wherein the first temperature control section is rectangular frame-like in shape.

(4) The reaction treatment device as described in the above paragraph (2) or (3), wherein the group of reaction regions is disposed in a substrate, and the first temperature control section and the second temperature control section make contact with the substrate.

(5) The reaction treatment device as described in any of the above paragraphs (2) to (4), wherein edges of an outer peripheral part and/or an inner peripheral part of a frame body portion of the first temperature control section are each provided with a single or a plurality of cutouts.

(6) The reaction treatment device as described in the above paragraph (5), wherein the cutout or cutouts are provided in corners of the outer peripheral part and/or in central parts of the edges of the inner peripheral part.

(7) The reaction treatment device as described in any of the above paragraphs (1) to (6), wherein the reaction region serves as a reaction site for a nucleic acid amplification reaction, and the reaction treatment device further includes: an irradiation section configured to irradiate the reaction region with light; and a detection section configured to detect light coming from the reaction regions.

(8) The reaction treatment device as described in the above paragraph (1), wherein the temperature control section is flat plate-like in shape and has a light-transmitting opening section at a part corresponding to each reaction region in the group of reaction regions.

(9) The reaction treatment device as described in the above paragraph (8), wherein the group of reaction regions is disposed in a substrate, and the temperature control section having the opening section makes contact with the substrate.

(10) The reaction treatment device as described in the above paragraph (8) or (9), wherein the temperature control section having the opening section is disposed between the substrate and a heat insulating section which restrains release of heat from the temperature control section.

(11) The reaction treatment device as described in any of the above paragraphs (8) to (10), wherein the temperature control section having the opening section has the opening section formed in a light-screening body, and the reaction treatment device further includes an irradiation section configured to irradiate the reaction regions with light and a detection section configured to detect light coming from the reaction regions.

(12) The reaction treatment device as described in any of the above paragraphs (8) to (11), including two above-mentioned temperature control sections having the opening section, the two temperature control sections being disposed opposite to each other, with the reaction region group therebetween.

(13) A reaction treatment method wherein temperature of an outer peripheral edge part of a group of reaction regions is controlled by a temperature control section disposed at least at the outer peripheral edge part, whereby temperature of the reaction region group is controlled.

(14) The reaction treatment method as described in the above paragraph (13), wherein the temperature control section is a first temperature control section disposed at the outer peripheral edge part of the reaction region group, the first temperature control section and a planar second temperature control section are disposed opposite to each other, with the reaction region group having a plurality of reaction regions therebetween, and the first temperature control section and the planar second temperature control section cooperate with each other so as to control temperature of the reaction region group.

(15) The reaction treatment method as described in the above paragraph (14), wherein a single or a plurality of cutouts are provided in an outer peripheral part and/or an inner peripheral part of a frame body portion of the first temperature control section so as to suppress local heating.

(16) The reaction treatment method as described in the above paragraph (13), wherein temperature of the reaction region group is controlled by suppressing local heating in the reaction region group while controlling temperature of the outer peripheral edge part by a flat plate-like temperature control section which has a light-transmitting opening section at a part corresponding to each reaction region in the reaction region group.

### EXAMPLES

### Experimental Example 1

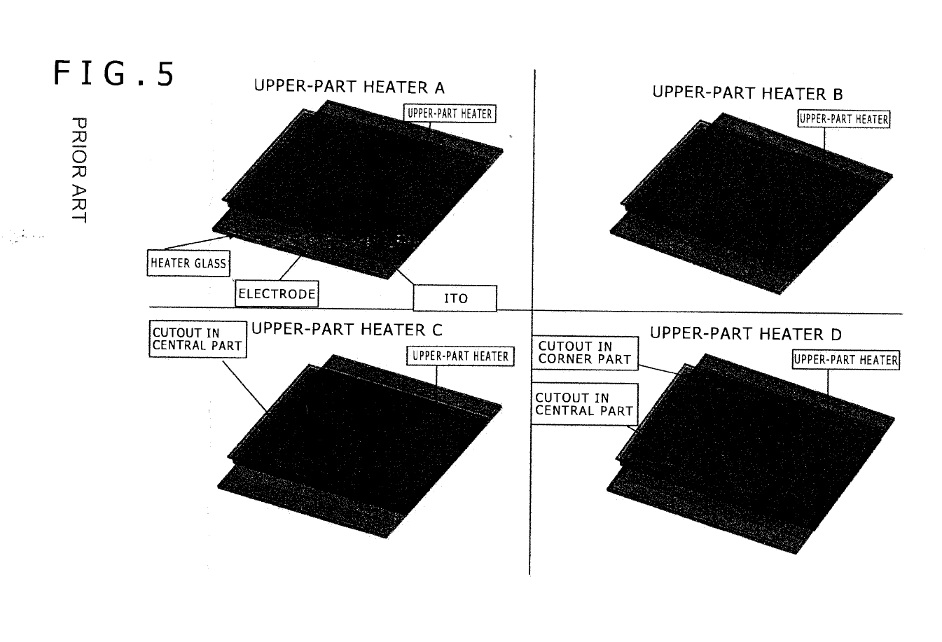

Analysis Models as Shown in FIGS. 4 and 5 were Fabricated.

Specifically, a microchannel chip is clamped, in a sandwiched manner, between a planar ITO heater (lower-surface heater) and a rectangular frame-shaped heater (upper-part heater). A structure is adopted in which the heat generating section is thermally insulated from the casing.

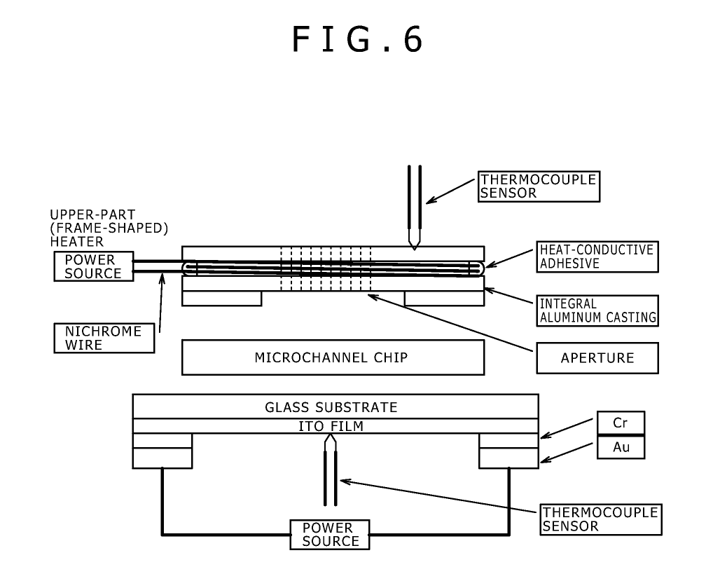

The rectangular frame-shaped heater is composed of an aluminum casting, which is counter sunk in an angular shape in a central portion thereof, and is provided with a notch in an outer peripheral surface thereof like a bobbin. A nichrome wire is wound along the peripheral notch. The nichrome wire and the notch receiving the nichrome wire were sealed with a heat-conductive adhesive so as to prevent a heat distribution from being generated due to differences in the manner of winding. It is the lower-surface ITO heater in contact with the lower surface of the chip that has the role of compensating for the heat released from the upper side of the chip, while being put to heat generation at a fixed output, and that controls the temperature of the chip. The ITO heater has a structure in which rectangular sheet-shaped Cr/Au electrodes are sputtered on both ends of a substrate provided thereon with ITO film by sputtering, with a wire connected to Au by soldering (see FIG. 6).

The ITO heater is equipped with a thermocouple sensor in such a manner as not to interfere with the optical system. The upper-part heater is also equipped with a thermocouple sensor in an embedded state. The outputs of the heaters are controlled by PID control (involving feedback from both the sensors) so that the inside of the microchip can be uniformly heated.

Temperature difference simulation with respect to the inside of the microchip reaction site well in each analysis model was conducted using STREAM available from Software Cradle Co., Ltd., while using non-steady three-dimensional heat conduction analysis as a computation system. The analytical results are set forth in Table 1 below.

TABLE 1

| | | Lower- | Lower- | Lower- | Lower- |
|---|---|---|---|---|---|
| | Lower- | surface | surface | surface | surface |
| | surface | heater + | heater + | heater + | heater + |
| | heater | upper-part | upper-part | upper-part | upper-part |
| | only | heater A | heater B | heater C | heater D |
| Well center | 2.77° C. | 3.61° C. | 0.61° C. | 0.14° C. | 0.14° C. |
| temperature | | | | | |
| difference | | | | | |
| MAX − | 4.24° C. | 4.45° C. | 1.23° C. | 0.77° C. | 0.76° C. |
| MIN, | | | | | |
| in effective | | | | | |
| surface of | | | | | |
| microwell | | | | | |

As shown in Table 1, it was verified that, owing to the structure in which the upper-part heater is rectangular frame-like in shape, it is possible to obviate a situation in which local heating would occur in a central area of the microchip because there are fewer heat release paths in the central area than in the peripheral area (see FIG. 4).

In addition, it was also confirmed that, owing to the structure in which the upper-part heater is provided with cutouts so that the heater does not make contact with chip parts where local heating is liable to occur, it is possible to more securely prevent local heating from occurring at the peripheral portions of the chip (see FIG. 4).

### Experimental Example 2

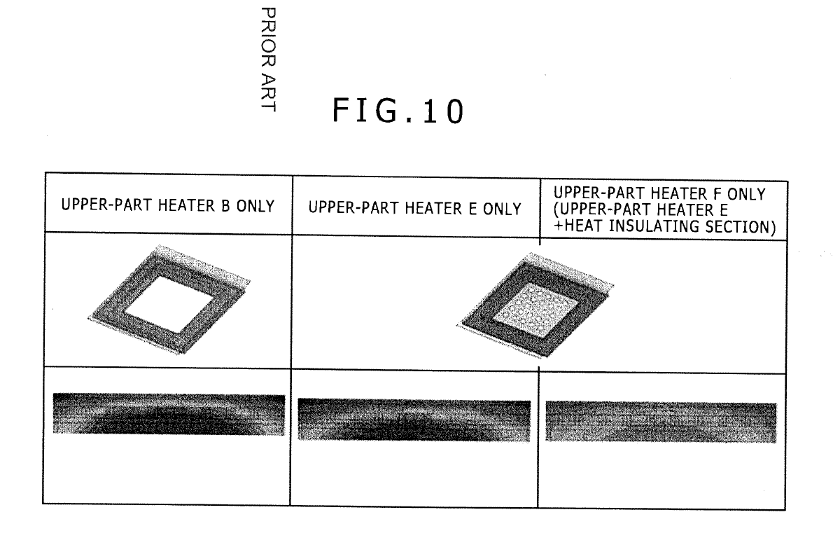

In Addition, Analysis Models as Shown in FIG. 10 were Fabricated.

Specifically, only an upper-part heater was disposed on a microchannel chip, without using any lower-surface heater. As the upper-part heater, there were used upper-part heaters E and F as well as the upper-part heater B that was used in Experimental Example 1 above. The upper-part heater E was flat plate-like in shape and had opening sections at parts corresponding respectively to the reaction regions. The upper-part heater E was fabricated by use of an aluminum casting with a nichrome wire wound around at an outer peripheral part of the reaction region group 2A, as described in Experimental Example 1 above. The upper heater F was fabricated by disposing polycarbonate as a heat insulating section on the upper surface of the upper-part heater E.

Temperature difference simulation with respect to the inside of the microchip reaction site well in each analysis model was carried out in the same manner as in Experimental Example 1. The analytical results are set forth in Table 2 below.

TABLE 2

| | Upper-part | Upper-part | Upper-part |
|---|---|---|---|
| | heater B only | heater E only | heater F only |
| Well center | 3.03° C. | 2.50° C. | 1.45° C. |
| temperature difference | | | |
| MAX − MIN, in effective | 4.26° C. | 3.72° C. | 1.19° C. |
| surface of microwell | | | |

As shown in Table 2, it was verified that, in the case of only the upper-part heater, the provision of the upper-part heater with the opening sections corresponding to the reaction regions restrains local heating from occurring in a central area of the microchip (see FIG. 10). It was also confirmed that in the case of the upper-part heater F in which the upper-part heater having the opening sections was provided with the heat insulating section, local heating in the central area of the microchip can be hampered more securely, and the maximum temperature difference within the reaction site well can be reduced (see FIG. 10).

Each of the reaction treatment devices according to embodiments of the present disclosure makes it possible to perform temperature control easily and accurately, whereby reaction detection accuracy and working efficiency are also enhanced. Moreover, since it is unnecessitated to take into account the method of arranging the reaction regions, the degree of freedom in designing the reaction region group in the substrate is enhanced. In addition, since the frame-shaped temperature control section is adopted, the device as a whole can be reduced in size, particularly, made into a thin handy type. Further, the reaction treatment device in the present disclosure is attended by little scattering of reaction among the reaction regions, so that it is advantageous to use the reaction treatment device as a nucleic acid amplification reaction device that is desired to have a high detection accuracy. Also, fluorescence detection can also be appropriately carried out with the reaction treatment device.

It should be understood by those skilled in the art that various modifications, combinations, sub-combinations and alterations may occur depending on design requirements and other factors in so far as they are within the scope of the appended claims or the equivalents thereof.

The application is claimed as follows:

1. A reaction treatment device comprising a temperature control section which controls temperature of an outer peripheral edge part of a group of reaction regions.

2. The reaction treatment device according to claim 1,
wherein the temperature control section is a first temperature control section disposed at the outer peripheral edge part of the group of reaction regions,
the reaction treatment device includes
a reaction temperature control section including
the first temperature control section and
a planar second temperature control section, and
the first temperature control section and the second temperature control section are disposed opposite to each other, with the reaction region group therebetween.

3. The reaction treatment device according to claim 2, wherein the first temperature control section is rectangular frame-like in shape.

4. The reaction treatment device according to claim 2,
wherein the group of reaction regions is disposed in a substrate, and
the first temperature control section and the second temperature control section make contact with the substrate.

5. The reaction treatment device according to claim 4, wherein edges of an outer peripheral part and/or an inner peripheral part of a frame body portion of the first temperature control section are each provided with a single or a plurality of cutouts.

6. The reaction treatment device according to claim 5, wherein the cutout or cutouts are provided in corners of the outer peripheral part and/or in central parts of the edges of the inner peripheral part.

7. The reaction treatment device according to claim 2,
wherein the reaction region serves as a reaction site for a nucleic acid amplification reaction, and
the reaction treatment device further comprises:
an irradiation section configured to irradiate the reaction regions with light; and
a detection section configured to detect light coming from the reaction regions.

8. The reaction treatment device according to claim 1, wherein the temperature control section is flat plate-like in shape and has a light-transmitting opening section at a part corresponding to each reaction region in the group of reaction regions.

9. The reaction treatment device according to claim 8,
wherein the group of reaction regions is disposed in a substrate, and
the temperature control section having the opening section makes contact with the substrate.

10. The reaction treatment device according to claim 9, wherein the temperature control section having the opening section is disposed between the substrate and a heat insulating section which restrains release of heat from the temperature control section.

11. The reaction treatment device according to claim 10,
wherein the temperature control section having the opening section has the opening section formed in a light-screening body, and
the reaction treatment device further comprises
an irradiation section configured to irradiate the reaction regions with light and
a detection section configured to detect light coming from the reaction regions.

12. The reaction treatment device according to claim 11, comprising
two said temperature control sections having the opening section, the two temperature control sections being disposed opposite to each other with the reaction region group therebetween.

13. A reaction treatment method wherein temperature of an outer peripheral edge part of a group of reaction regions is controlled by a temperature control section disposed at least at the outer peripheral edge part, whereby temperature of the reaction region group is controlled.

14. The reaction treatment method according to claim 13,
wherein the temperature control section is a first temperature control section disposed at the outer peripheral edge part of the reaction region group, and
the first temperature control section and a planar second temperature control section are disposed opposite to each other, with the reaction region group therebetween, and the first temperature control section and the planar second temperature control section cooperate with each other so as to control temperature of the reaction region group.

15. The reaction temperature method according to claim 14, wherein a single or a plurality of cutouts are provided in an outer peripheral part and/or an inner peripheral part of a frame body portion of the first temperature control section so as to suppress local heating.

16. The reaction treatment method according to claim 13, wherein temperature of the reaction region group is controlled by suppressing local heating in the reaction region group while controlling temperature of the outer peripheral edge part by a flat plate-like temperature control section which has a light-transmitting opening section at a part corresponding to each reaction region in the reaction region group.
